(19) United States
(12) Patent Application Publication
(10) Pub. No.: US 2010/0228232 A1
(43) Pub. Date: Sep. 9, 2010

(54) Catheter flushing assembly

(76) Inventors: Loay Salman, Miami, FL (US); Arif Asif, Miami, FL (US); Stephen R. Ash, Lafayette, IN (US)

Correspondence Address: MALLOY & MALLOY, P.A., 2800 S.W. Third Avenue, Historic Coral Way, Miami, FL, 33129, US

(21) Appl. No.: 12/658,899

(22) Filed: Feb. 16, 2010

Related U.S. Application Data
(60) Provisional application No. 61/153,638, filed on Feb. 18, 2009.

(51) Int. Cl. A61M 25/16

(52) U.S. Cl. 604/537

(57) ABSTRACT

An assembly for flushing at least the primary flow lines associated with a catheter, such as an intravascular catheter, comprising first and second flush ports connected in fluid communicating relation with a different one of said primary flow lines. A primary flow restrictor assembly includes two safety flow restrictors, disposed at a main hub of the catheter and the primary flow lines, and being structured to regulate fluid flow between the primary flow lines and the catheter. A flushing orientation is established when the two safety flow restrictors are concurrently disposed in a closed position, thereby establishing fluid flow between the primary flow lines and corresponding ones of the first and second flush ports, wherein one-way valves associated with each of the flush ports facilitate a concurrent path of flushing fluid from said primary flow lines and towards, out and through the first and second flush ports, to the external environment. Consequently, the flushing assembly of the present invention can prevent the introduction of flushing fluid, along with any bacteria or other collection of material carried by it, into a patient's bloodstream.

### CLAIM OF PRIORITY

The present application is based on and a claim of priority is made under 35 U.S.C. Section 119(e) to a provisional patent application that is currently pending in the U.S. Patent and Trademark Office, namely, that having Ser. No. 61/153,638 and a filing date of Feb. 18, 2009, the entirety of which is incorporated herein by reference.

### BACKGROUND OF THE INVENTION

1. Field of the Invention

This invention relates to catheter technology and more specifically, to a flushing assembly which can be operated to concurrently flush a plurality of primary flow lines that are normally disposed in fluid communication with a working catheter. Use of the flushing assembly decreases the risk of many types of infections typically associated with catheter use, by establishing a flushing orientation comprising independent and concurrent paths of flushing fluid-flow from each of the primary flow lines, towards, through and out of separate flush ports, each of which is associated with a different one of the plurality of primary flow lines.

2. Description of the Related Art

The use of catheters for a variety of different medical procedures is well known and commonly practiced in the field of medicine. More in particular, a working catheter, dependent upon its intended use, is applied to a patient and operatively structured to carry different fluids including, but not limited to, blood, urinary fluids, biliary fluids, etc. By way of example, intra-vascular catheters may be applied and/or inserted beneath the skin of a patient with chronic kidney disease, then moved into his or her bloodstream, into or through the central veins and into the heart, where it can be used to recycle and process the patient's blood with the aid of a dialysis machine. This procedure, known as hemodialysis, typically involves the placement of a catheter in a patient's body and bloodstream and leaving it there for a relatively long period of time because the patient is suffering from kidney failure, such that the treatment procedure or sessions must be ongoing, e.g., three days a week for months or longer at a time. Thus, the catheter is in place on or in the patient for a relatively long period of time in order to provide a ready means for vascular access into his/her bloodstream. A particular technique is typically followed for placement of such catheters, which involves creating a small incision in the patient's skin, often near the shoulder and neck area; puncturing into the bloodstream and advancing a double-lumen catheter into the central veins and the heart, where the catheter can remain in the patient safely for a relatively long period of time. The opposite or external end of the catheter can remain somewhat exposed on the patient's skin, and while protected, serves as a port for providing access to his/her vascular system.

Regardless of the specific application of the catheter assembly, it has been found to be important for the interior lumens of such devices to be kept clean and/or otherwise maintained, at least to the extent of limiting the introduction of bacteria into them and also, removing any bacterial presence and/or assuring that the passageways of the catheter lumens are free or at least substantially free from the collection or build-up of residue or clots and the like, on the interior surfaces thereof. It is universally recognized that the build-up of residue or bacterial formations leads to a possible interruption of fluid flow through the catheter, or of potentially grave danger, infection. Additionally, if there is limited or uneven fluid flow through the lumens of the catheter, it may delay the treatment time for a patient and/or recovery time, dependent upon the intended use of the catheter assembly. Such delays may, in turn, result in complications or possibly to a life threatening infection if harmful bacteria is present and/or is permitted to grow and colonize. Problems of this type are especially prevalent with catheters intended for longer periods of installation and use, such as those described above and commonly used for dialysis.

As a result, various components of a working catheter must be periodically “flushed” to ensure that any build up, such as that described above, is completely or at least substantially removed and that the presence of bacteria is greatly reduced, if not eliminated. In conventional fashion, the flushing of a catheter assembly involves the connection of a source of flushing fluid such as, but not limited to, a saline solution directed under pressure through the lumens of each of the two catheter ports. During the flushing procedure, the flushing fluid is then allowed to flush the catheter lumens and be deposited in the bloodstream. While this flushing procedure is commonly applied at the initiation of each dialysis session, a number of disadvantages and/or problems may be encountered. For instance, upon initiation of each dialysis treatment, and in accordance with standard practice, the hub of the catheter is opened manually. Such handling can introduce bacteria into both the catheter lumens and has been known to be a major factor in causing catheter related infections. The conventional standard flushing phase that comes next flushes bacteria and other constituents contained in the catheter into the bloodstream, thereby increasing the risk of catheter related infections.

Accordingly, it would be prudent to devise a system that would allow for an alternative way to flush the catheter, such that bacteria and other constituents could be flushed from at least a portion of the catheter to the external environment, instead of into the bloodstream of the patient.

In other words, there is a need in the medical profession for an efficient and effective assembly which facilitates the flushing of at least predetermined portions of a catheter, such as, but not limited to, the ingress and egress lines or other “primary flow lines” of an intravascular catheter, as generally set forth above. Further, if any such improved flushing assembly were developed, it would preferably include one or more structural and operative features which render it readily adaptive and effectively operable to accomplish the required or intended flushing procedure, without fear of inadvertently exposing the patient to the flushing fluid, in case a fluid or agent for cleaning bacteria, other than saline solution, is used and which might be toxic if infused into the bloodstream. If any such flushing assembly were developed, it would ideally include appropriately structured and disposed flush ports, associated with the primary flow lines to be flushed, in a manner which regulates the introduction and passage of the flushing fluid through the flow lines in a direction towards the flush ports and outwardly there-from, such that any collected residue and in particular, bacterial formations are removed in an effective, efficient and safe manner. Finally, it would also be ideal if any such proposed flushing assembly were capable of being adaptable for use with a variety of catheter structures, whether by being fixedly or removably associated with the catheter, and thereby, able to provide versatility in structure and operation to accomplish an efficient flushing procedure.

### SUMMARY OF THE INVENTION

The present invention is intended to present a solution to these and other needs which remain in this field of art, and as such, is directed to an assembly for flushing proximately disposed, primary flow lines associated with a working catheter, such as, but not limited to, an intravascular catheter. More specifically, the primary flow lines may comprise egress and ingress lines associated with the catheter, which are proximally disposed in a physically accessible location when the catheter is implanted. Further, the primary flow lines may be structured to be part of the catheter or an auxiliary structure connected to or otherwise associated with the catheter. In addition, the primary flow lines are associated with or operatively connected to a main hub or junction and thereby, disposed in fluid communication with the lumens of catheter embedded within the patient.

More specifically, the flushing assembly of the present invention includes a first flush port and a second flush port, each connected to a different one of the ingress and egress lines, which are synonymously referred to herein as the primary flow lines. A primary flow restrictor assembly, preferably comprising a plurality of two independently adjustable safety valves or alternatively, two safety flow restrictors which may be in the form of externally applied clamps or other appropriate flow restrictor structures. As such, the primary flow restrictor assembly is connected at the main junction or hub serving to interconnect the lumens of the catheter with the primary flow lines. Moreover, each of the safety valves or clamps is structured to be selectively disposed between an open position and a closed position. This feature facilitates the selective regulation of fluid flow from the primary flow lines into the working catheter, as well as regulating fluid communication between the first and second flush ports and the primary flow lines, as will be described in greater detail hereinafter. A secondary flow restrictor assembly comprises at least two clamps or other appropriate flow restrictors, independently operable and positioned, to isolate or establish fluid communication between the first and second flush ports and corresponding ones of the primary flow lines.

In use, a flushing orientation of the flushing assembly is established by concurrently disposing each of the two safety valves or safety clamps in a closed orientation, while removing or otherwise opening the clamps associated with the secondary flow restrictor assembly. This will define open, fluid communication between the primary flow lines and corresponding ones of the first and second flush ports. Flushing occurs by directing or “pushing” a flow of flushing fluid into the open ends of the primary flow lines, while the flushing assembly is in the aforementioned flushing orientation. As a result, a forced flow of flushing fluid will pass concurrently into and along each of the primary flow lines and there-from, into and through the corresponding first and second flush ports.

In at least one preferred embodiment, a one-way valve is connected at the exit end of each of the first and second flush ports, with each structured to allow the passage of the flushing fluid out of the flush ports through the corresponding exit ends thereof. Moreover, the one-way valves are further structured to prevent the passage of fluid into the flush ports through the exit ends from an exterior of the catheter or flushing assembly. Accordingly, the structural and operative features of the flushing assembly of the present invention facilitate the flushing of bacteria and other collected material from the primary flow lines in a direction towards the corresponding flush ports and outwardly therefrom, through corresponding exit ends associated with the flush ports.

After the flushing procedure, the clamps associated with the secondary flow restrictor assembly may be closed, thereby isolating fluid communication between the first and second flush ports and respective ones of the primary flow lines. In order to make the flushing procedure completely sterile, an appropriately dimensioned, sterile syringe can be inserted into the one-way valve located at the exit end of each of the flush ports in order to receive the fluid being flushed. After the catheter has been used, the traditional “locking” procedure may be applied utilizing a heparin or other appropriate solution, as is known in the art.

In addition to the above, and in an effort to decrease the risk of infection, the lumens of the catheter and/or the lumens of the flush ports can be filled with an antiseptic/antibiotic solution, if desired. Utilization of the flushing assembly in the intended manner, as generally set forth above, can be said to offer an additional benefit by preventing the antimicrobial solution (antibiotic/antiseptic) from entering the catheter and patient's blood stream. Seepage of the antibiotic/antiseptic solution into circulation is prohibited due to the cooperative structuring of the flush ports with the primary and secondary flow restrictor assemblies. As a result, antibiotic resistance and medication toxicity can be avoided. Additional features of at least one preferred embodiment of the present invention is the connection of the two safety valves at the main junction or hub connecting the primary flow lines to the lumens of the remainder of the catheter. This location serves to eliminate “dead space” in the area of the path of fluid flow of the flushing fluid, during the flushing procedure. Therefore, the two safety valves are disposed and structured to selectively and concurrently close the connection of the primary flow lines to the lumens of the catheter, and accordingly, to the patient when the catheter is not in use. The selective manipulation of the safety valves or other safety flow restrictors also provides for the holding and maintaining of antiseptic, antibiotic solution in the lumens of the flush ports, as well as the primary flow lines when the catheter is not being used.

The flushing assembly of the present invention will now be described in greater detail hereinafter in various preferred embodiments, and also, with specific reference to an intravascular catheter. It is pointed out, however, that the flushing assembly of the present invention can be utilized, with minimal or no structural modification, in conjunction with other long term use catheter structures including, oncology catheters, nutrition catheters, short and long term central line catheters, etc.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a front view in partial cutaway of one preferred embodiment of the flushing assembly of the present invention.

FIG. 2 is a front view in partial cutaway of yet another preferred embodiment of the flushing assembly of the present invention.

FIG. 3 is a front view of in partial cutaway of yet another preferred embodiment of the flushing assembly of the present invention.

FIG. 4 is a front view of yet another preferred embodiment of the flushing assembly of the present invention.

FIG. 5 is a front view of yet another preferred embodiment of the flushing assembly of the present invention.

FIG. 6 is a front view of yet another preferred embodiment of the flushing assembly of the present invention.

FIG. 7 is a front view of yet another preferred embodiment of the flushing assembly of the present invention.

FIG. 8 is a front view of yet another preferred embodiment of the flushing assembly of the present invention.

FIG. 9 is a front view in partial cutaway of another preferred embodiment of the present invention similar to but distinguishable from the embodiment of FIG. 8.

FIG. 10 is a front view in partial cutaway of yet another preferred embodiment of the present invention similar to but distinguishable from the embodiment of FIG. 9.

Like reference numerals refer to like parts throughout the several views of the drawings.

### DETAILED DESCRIPTION OF THE PREFERRED EMBODIMENT

While the invention is susceptible to various modifications and alternative forms, specific embodiments thereof have been shown by way of example in the attached drawings and will herein be described in detail. It should be understood, however, that there is no intent to limit the invention to the particular forms disclosed, but on the contrary, the intention is to cover all modifications, equivalents, and alternatives falling within the spirit and scope of the invention, as defined by this description and the appended claims.

Turning now to the accompanying drawings, the present invention is directed to a flushing assembly, generally indicated as 10, structured to flush at least the proximately located ingress and egress lines, hereinafter also referred to as “primary flow lines,” 12 and 14 associated with a catheter assembly 16. While the structural and operative versatility of the flushing assembly 10 facilitate its use with a variety of different catheter structures, the present invention is described hereinafter with specific, but not necessarily exclusive, reference to an intravascular catheter. In addition, the primary flow lines 12 and 14 are connected in fluid communication with the catheter assembly 16 by a hub and/or main junction 18.

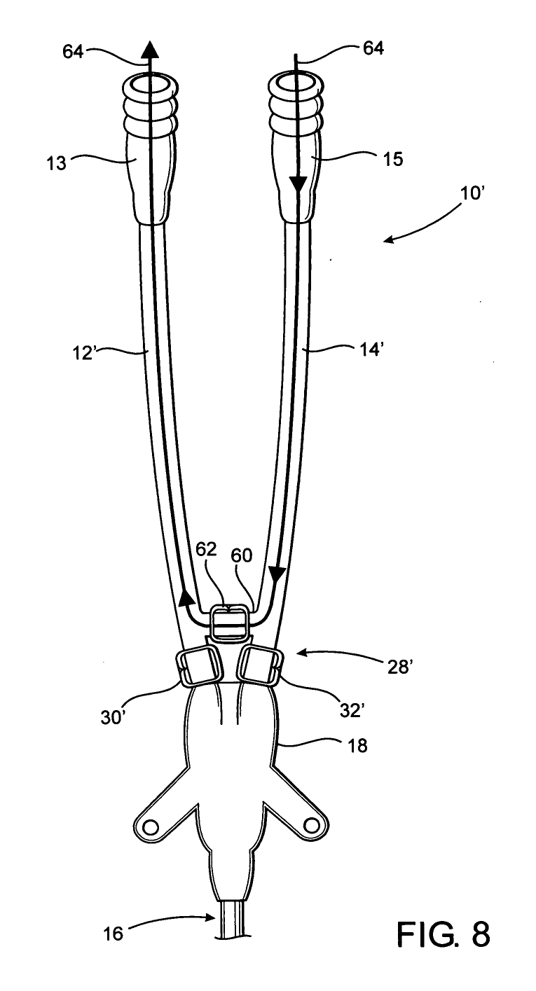

It is further emphasized that the primary flow lines 12 and 14, as well as other operative components associated with the flush assembly 10, may be considered in one preferred embodiment to be a fixed or integrated part of the catheter assembly 16. In other preferred embodiments, however, the primary flow lines 12 and 14 may be a supplementary component that may be effectively connected to the catheter assembly 16 and easily removed therefrom, as described for instance, with reference to the invention represented in FIGS. 8, 9 and 10.

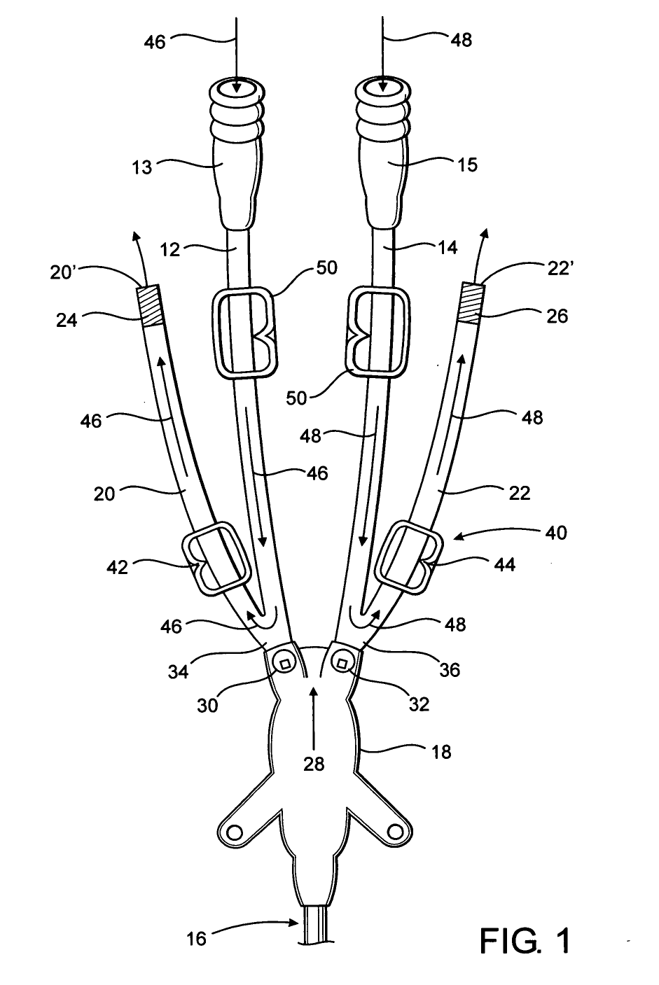

Turning now to FIG. 1, the flushing assembly 10 of this embodiment can be seen to include a first flush port 20 and a second flush port 22, each of which includes a substantially elongated configuration. Moreover, each of the flush ports 20 and 22 include a corresponding exit end 20′ and 22′ which have a one-way valve 24 and 26 connected in flow-regulating relation thereto. Also, end caps or injection caps (not shown) may be disposed in overlying or covering relation to the exit ends 20′ and 22′ as well as the corresponding one-way valves 24 and 26, when the flushing assembly 10 is not in use. Similarly, injection caps or end caps 13 and 15 may also be mounted on the open ends of the primary flow lines 12 and 14, as represented.

Still referring to FIG. 1, the flushing assembly 10 further includes a primary flow restrictor assembly, generally indicated as 28, comprising a plurality of at least two, selectively adjustable safety valves 30 and 32. The safety valves 30 and 32 are preferably either fixedly connected to or integrally incorporated within the main junction or hub 18. Also, the safety valves are disposed adjacent junctions 34 and 36 defining fluid communicating connections between the primary flow lines 12 and 14 and corresponding ones of the flush ports 20 and 22. As described in greater detail hereinafter, the specific location or placement of the flush ports 20 and 22 relative to corresponding ones of the flow lines 12 and 14 may vary. However, in one or more preferred embodiments the fluid communicating junctions 34 and 36 preferably include a Y-shaped structure or configuration. As such, both the primary flow lines 12 and 14, as well as the first and second flush ports 20 and 22, may be disposed in fluid interconnecting relation to one another at a location immediately adjacent to the primary flow restrictor assembly 28, specifically including the adjustable, safety valves 30 and 32. This cooperative, adjacent disposition of the adjustable safety valves 30 and 32 eliminates or reduces the possibility of any “dead space” which may interfere with the intended fluid flow throughout the flushing assembly 10 and/or the primary flow lines 12 and 14.

As set forth above, the two safety valves 30 and 32 are selectively adjustable and capable of being independently or collectively oriented between an open flow position and a closed flow position. When in the open position, the safety valves 30 and 32 establish fluid communication between the primary flow lines 12 and 14 and the main hub or junction 18, as well as the lumens of the catheter 16. In contrast, selective orientation of the safety valves 30 and 32 into a closed position will isolate fluid communication between the primary flow lines 12 and 14 and the main junction or hub 18, as well as the catheter 16.

Still referring to FIG. 1, additional structural and operative features associated with the flushing assembly 10 are the provision of a secondary flow restrictor assembly, generally indicated as 40. Moreover, the secondary flow restrictor assembly 40 includes two, independently operable clamps or other appropriate flow restrictors 42 and 44, each positioned in flow restricting relation to a different, corresponding one of the flush ports 20 and 22. When in the form of a clamp or other removable flow restrictor structure, each of the flow restrictors 42 and 44 may be selectively removed from or applied to the corresponding flush ports 20 and 22. Therefore, as should be apparent, a predetermined positioning or adjusting of the primary flow restrictor assembly 28 and the secondary flow restrictor 40 will result in the flushing assembly 10 assuming a “flushing orientation”.

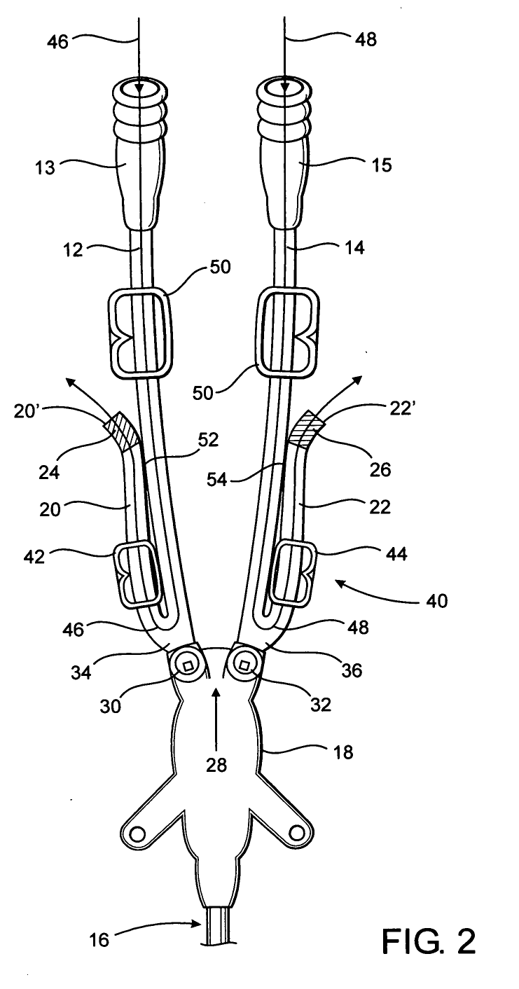

More specifically, and as at least partially set forth above, the adjustment of each of the safety valves 30 and 32 to a closed position will isolate fluid communication and restrict fluid flow between the primary flow lines 12 and 14 and the hub 18 and lumens of the catheter 16. Concurrently, the flow restrictors 40 and 42 of the secondary flow restrictor assembly 40 may be removed or otherwise adjusted, so as to be opened and thereby, establish fluid flow and fluid communication between the primary flow lines 12 and 14 and corresponding ones of the flush ports 20 and 22. Once the flushing assembly 10 is in the aforementioned “flushing orientation” then a flushing fluid such as, but not limited to, a saline solution can be forced or “pushed” into the primary flow lines 12 and 14, through the end or ejector caps 13 and 15. Flow of the flushing fluid will continue along the length of the respective primary flow lines 12 and 14 into, through, out of and away from corresponding ones of the flush ports 20 and 22. The direction of flushing fluid flow is indicated by corresponding directional arrows 46 and 48 in FIGS. 1 and 2. Accordingly, the forced flow of flushing fluid will be directed through the interior of the primary flow lines 12 and 14 towards the corresponding flush ports 20 and 22. The presence and structuring of the one-way check valves 24 and 26, each associated with a corresponding one of the exit ends 20′ and 22′ will facilitate the passage of the flushing fluid through and out of the open ends 20′ and 22′. Therefore, it should be apparent that the one-way valves 24 and 26 are structured to facilitate fluid flow out from the interior of the flush ports 20 and 22 to the external environment, such as to an appropriate collection facility, or if desired, a sterile, sufficient capacity syringe. In contrast, one-way valves 24 and 26 prevent the entrance of fluid into the interior of the corresponding flush ports 20 and 22 from an exterior of the flushing assembly 10 through the exit ends 20′ and 22′ respectively. As set forth above, in order to make the flushing procedure completely sterile, a sterile, empty syringe of sufficient capacity may pass into and through the one-way valves 24 and 26, in order receive the fluid being flushed from the interior of the flush ports 20 and 22.

As represented in the embodiment of FIG. 1, as well as other preferred embodiments represented in the accompanying Figures, additional flow restrictor structures, as at 50, may be selectively and/or removably secured to the primary flow lines 12 and 14 and/or other operative components associated with flushing assembly 10.

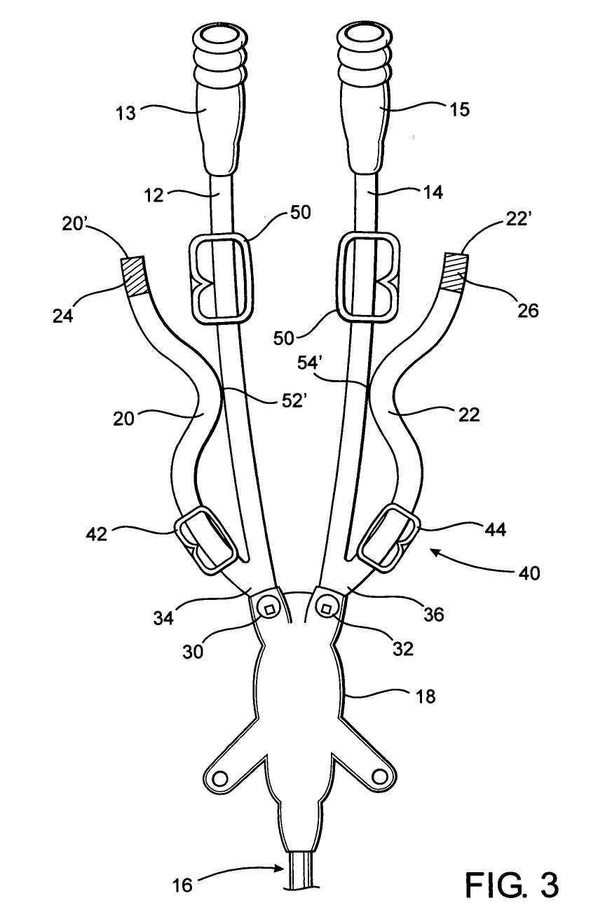

As set forth above, the location and/or disposition of the flush ports 20 and 22, relative to corresponding ones of the primary flow lines 12 and 14, may vary. Therefore, with primary reference to FIGS. 2 and 3, each of the flush ports 20 and 22 may be fixedly secured to corresponding ones of the flush lines 12 and 14, such as at 52 and 54. The respective attachments or connections 52 and 54 preferably extend along at least the majority of the length of the flush ports 20 and 22 and thereby, serve to facilitate the relatively stable placement/location of the flush ports 20 and 22 relative to corresponding ones of the primary flow lines 12 and 14.

As represented in the additional preferred embodiment of FIG. 3, attachment of the first and second flush ports 20 and 22 to corresponding ones of the primary flow lines 12 and 14 occurs at a point or location 52′ and 54′ along the length of but intermediate to the opposite ends of the first and second flush ports 20 and 22. As such, remaining lengths of each flush port 20 and 22 remain disconnected, in spaced relation from corresponding ones of the primary flow lines 12 and 14. As a result, the stable positioning or placement of the flush ports 20 and 22 is established, while providing an enhanced versatility to the manipulation and/or placement of the flush ports 20 and 22 and in particular the exit ends thereof 20′ and 22′.

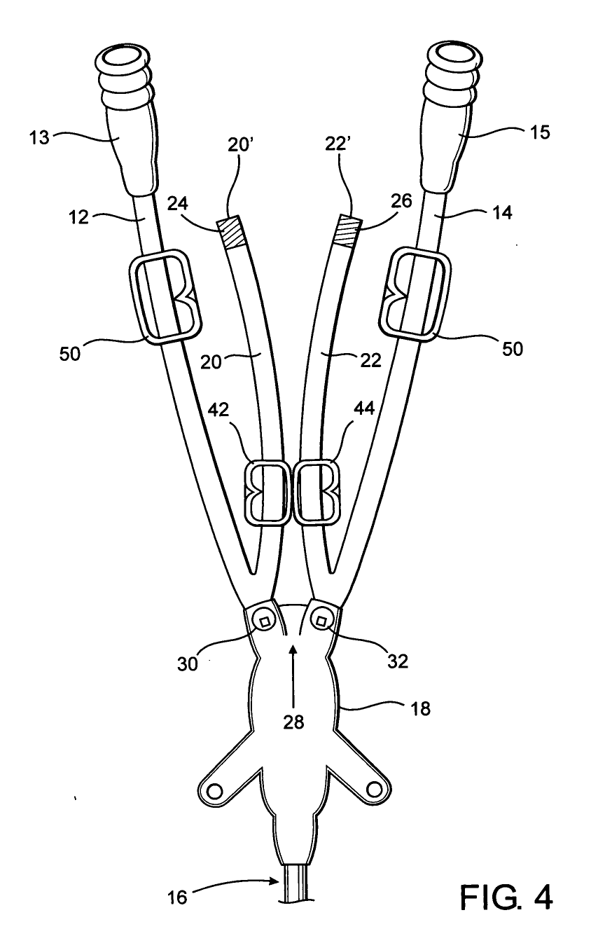

The versatility of the flushing assembly 10 is further represented in FIG. 4 wherein the first and second flush ports 20 and 22 are connected to corresponding ones of the primary flush lines 12 and 14, but distinguishable from the embodiments of FIGS. 1-3 by being located substantially on the interior of the corresponding primary flow lines 12 and 14. Moreover, in each of the preferred embodiments of FIGS. 1 through 4, a fluid communicating connection is established between the flush ports 20 and 22 and corresponding ones of the primary flow lines 12 and 14 by the substantially Y-shaped junction 34 and 36. Further, each of these preferred embodiments include the two safety valves 30 and 32, of the primary flow restrictor assembly 28, being disposed immediately adjacent or contiguous to the Y-shaped junctions 34 and 36. Moreover, in the embodiment of FIG. 4, the secondary flow restrictor assembly including the flow restrictor structures or clamps 42 and 44 are also located substantially interiorly of the corresponding primary flow lines 12 and 14. Common structural features of the flush ports 20 and 22 are included in the embodiment of FIG. 4 comprising the exit openings 20′ and 22′ each being associated with a different one-way valve 24 and 26 disposed and structured to restrict fluid flow into the interior of the flush ports 20 and 22 through the respective open ends 20′ and 22′.

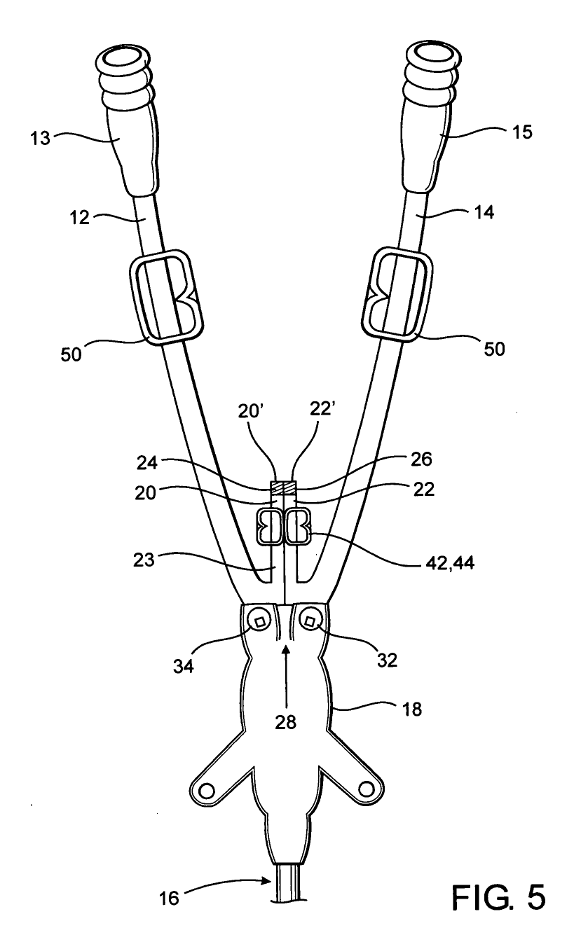

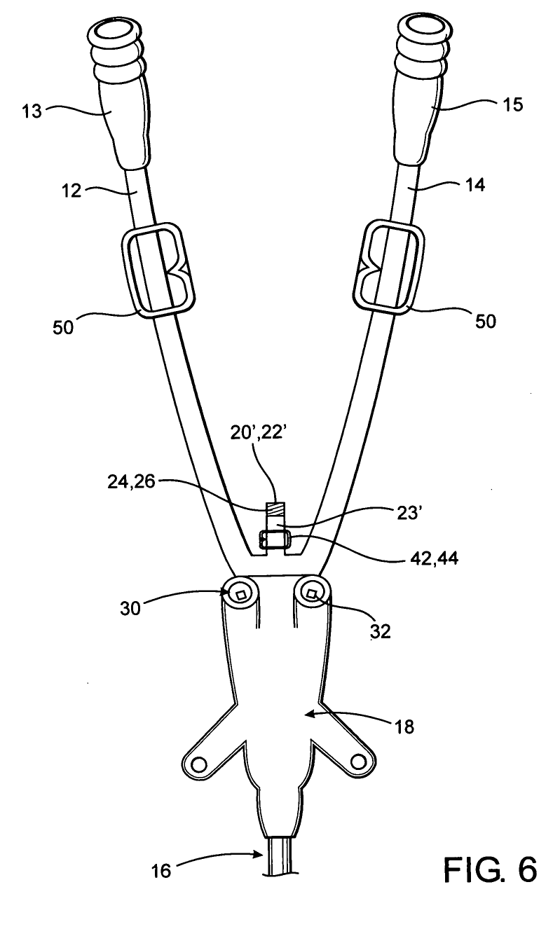

Yet additional preferred embodiments are represented in FIGS. 5 and 6. More specifically, FIG. 5 is directed to a flushing assembly, generally indicated as 10, including the primary flow lines 12 and 14 interconnected by fluid communicating Y-shaped junctions 34 and 36 to the flush ports 20 and 22. However, the flush ports 20 and 22 are located laterally between the corresponding primary flow lines 12 and 14, and further, are constructed into a common conduit 23. As such, the conduit or line 23 is structured to include a “dual-lumen” configuration. Therefore, each of the flush ports 20 and 22 are defined by a different one of the interior dual-lumen construction, wherein both of the interior lumens of flush ports 20 and 22 are integrated into the interior of a common conduit or line 23. As with the embodiments set forth in FIGS. 1-4, each of the flush ports 20 and 22 include an exit end 20′ and 22′ being associated with a one-way valve structure 24 and 26. Also, the secondary flow restrictor assembly of this embodiment comprises either a common flow restrictor 42, 44 or individual, separately operable flow restrictors 42 and 44.

Turning now to the additional preferred embodiment of FIG. 6, it can be noted that certain structural features thereof are similar to that represented in FIG. 5. More specifically, each of the primary flow lines 12 and 14 are connected to establish fluid communication between corresponding ones of first and second flush ports 20 and 22. However, in this embodiment the first and second flush ports 20 and 22 are defined by a common lumen integrated within the interior of a single conduit or line 23′. The secondary flow restrictor assembly 40 includes a common flow restrictor structure, also designated 42, 44 for purposes of clarity. Similarly, the common lumen of the line 23′ includes an exit end 20′, 22′ associated with a single one-way valve, designated 24, 26.

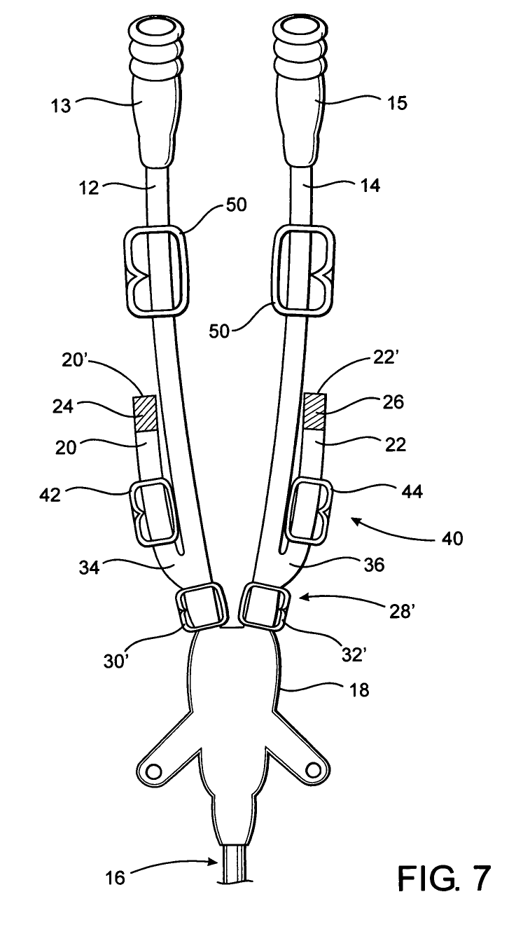

Turning now to FIG. 7, the flushing assembly is illustrated in yet another preferred embodiment which is operationally similar to the embodiments of FIGS. 1-6, and yet structurally distinguishable there-from, at least by a modification of the primary flow restrictor assembly, generally represented as 28′. More specifically, the primary flow restrictor assembly 28′ includes two independently operable safety flow restrictors 30′ and 32′, each disposed immediately adjacent to and/or as part of the main junction or hub 18. Each of the safety flow restrictors 30′ and 32′ will ideally comprise a clamp structure, selectively disposed in either a closed position or an opened position. More specifically, when in a closed position, the safety flow restrictor clamps 30′ and 32′ are disposed in clamping engagement on and/or with the lines disposed adjacent to the hub 18, as represented in FIG. 7. In contrast, when in an opened position, the safety flow restrictors or clamps 30′ and 32′ may be removed from the lines or conduits adjacent to the hub 18, or otherwise manipulated or disposed, so as not to interrupt flow between the primary flow lines 12 and 14 and the catheter 16. Accordingly, when selectively disposed in a closed position, the safety flow restrictors 30′ and 32′ serve to isolate fluid communication between the interior of the primary flow lines 12 and 14, and the main junction or hub 18, as well as the lumens associated with the catheter assembly 16. Similarly, when the safety flow restrictors 30′ and 32′ are disposed in a closed position, the flow restrictors 42 and 44 may be removed or disposed in an open flow position, thereby facilitating the flushing assembly 10, assuming the aforementioned flushing orientation. Similar to the embodiments of FIGS. 1-6, the path or flow or flushing fluid will be pushed from the interior of the primary flow lines 12 and 14 to and through the corresponding Y-shaped junctions 34 and 36, and into and through the corresponding flush ports 20 and 22 to the external environment, and not into the patient's bloodstream. Thus, the flushing fluid, along with bacteria and any other collected material, will exit the respective flush ports 20 and 22 through the exit ends 20′ and 22′, wherein such exiting flow of flushing fluid is facilitated by the provision of the one-way valves 24 and 26.

Yet another preferred embodiment of the present invention is represented in FIG. 8, wherein the flushing assembly 10′ utilizes the interior lumens of the primary flow lines 12′ and 14′ as the path of flow, along which the flushing fluid is forced in order to properly flush the interior of the primary flush lines 12′ and 14′. In this embodiment, the flushing assembly 10′ may at least partially comprise an “H-shaped” configuration, wherein a bridging line 60 allows for fluid communication and interconnection between the interiors of the primary flow lines 12′ and 14′. Further, the secondary flow restrictor assembly is represented by a single flow restrictor 62, which may be in the form of a clamp or other appropriate flow restrictor structure. Somewhat similar to the embodiment of FIG. 7, the primary flow restrictor assembly 28′ includes the independently operable flow restrictor structures, clamps and/or valves 30′, 32′ disposed in flow restricting relation between the primary flow lines 12′ and 14′ and the hub 18, as well as the lumens of the catheter assembly 16. Accordingly, in this embodiment, disposition of the safety flow restrictors 30′ or 32′ in a closed position and a concurrent opening or removal of the flow restrictor 62 establishes a path of flushing fluid flow generally indicated as 64. More specifically, the path of flushing fluid flow 64 includes an entry into and passage along one of the primary flow lines, such as at 14′ and into and through the bridging line or conduit 60. The path of flushing fluid flow continues into and along the opposite of the two primary flow lines, such as at 12′, from which it exits. As should be apparent, the opening of the safety flow restrictors 30′ and 32′ and a closing of the flow restrictor 62 serves to establish conventional or normal fluid flow between the primary flow lines 12′ and 14′, the main junction or hub 18 and the lumens of the catheter assembly 16.

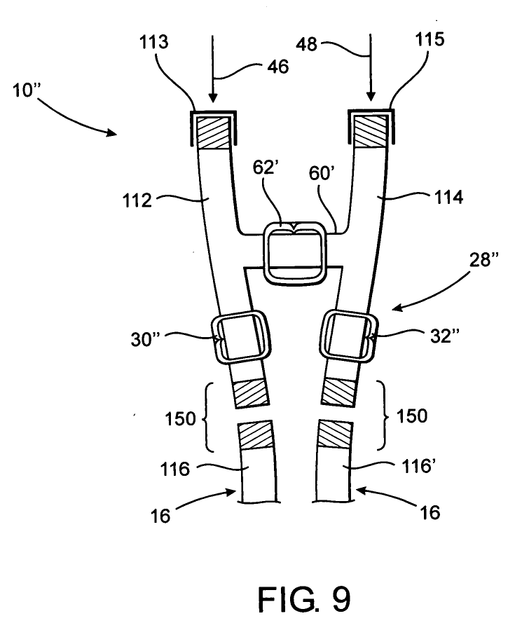

FIG. 9 illustrates yet another preferred embodiment of the flushing assembly, and is generally indicated as 10″. This embodiment of the flushing assembly 10″ is operationally similar to the embodiment of FIG. 8, wherein primary flow lines 112 and 114 are interconnected by a bridge line or conduit 60′. Fluid flow and/or fluid communication between the primary flow lines 112 and 114 is regulated by the opening or closing of the flow restrictor 62′ which, in terms of the embodiments of FIGS. 1-7, may be considered similar in operation to the secondary flow restrictor assembly 40. As also represented in FIG. 9, the primary flow lines 112, 114 as well as the bridging line 60′ is removably connected to correspondingly disposed and operative lumens 116 and 116′ of the remainder of the catheter 16 auxiliary flow lines 12′ and 14′ which may duplicate or take the place of the primary flow lines 12 and 14, as represented in FIGS. 1-7. Similarly, the primary flow restrictor assembly is generally indicated as 28″ and includes appropriately structured and independently operative clamps or valves 30″ and 32″ connected in flow restricting relation to the flow lines 112 and 114. An appropriate connection assembly 150 is used to removably attach the corresponding ends of the primary flow lines 112 and 114 to the catheter lumens 116 and 116′ which are directly associated with the catheter assembly 16. Therefore, flushing orientation of the flushing assembly 10″ is accomplished by closing of the flow restrictors 30″ and 32″, while removing or opening the flow restrictor 62′. The path of flushing fluid flow will be similar to that represented in FIG. 8, in that it will be forced into one of the ends 113 or 115 and along the length of the corresponding flow line 112 or 114, passing through the bridge line 60′ and thereafter passing into and exit from the opposite end 113 or 115 of the flow corresponding flow line 112 or 114. As also specifically represented in FIG. 9, the primary flow lines 112 and 114 and the bridging conduit 60, collectively define a substantially H-shaped configuration. Moreover, the H-shaped configuration of the embodiment of the assembly 10″ of FIG. 9 is more pronounced than that of the assembly 10′ of the embodiment of FIG. 8, although the operation of these two embodiments is similar.

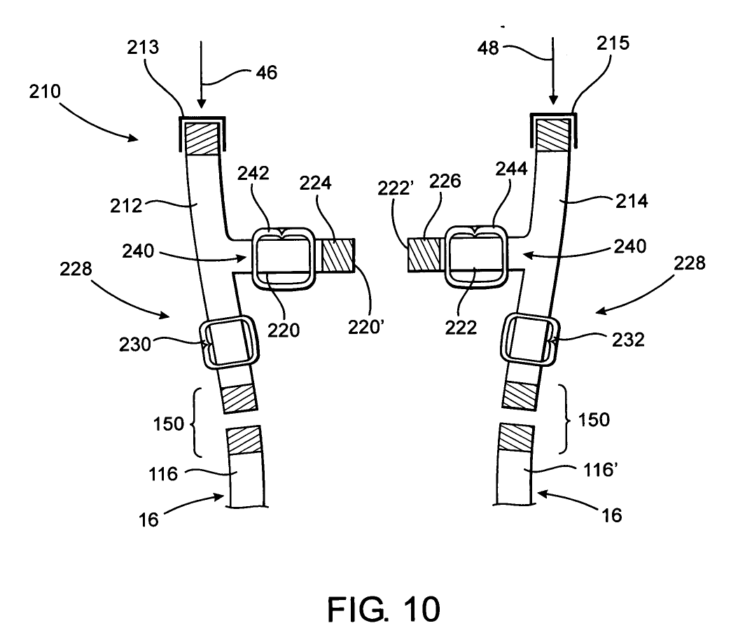

Yet another preferred embodiment is illustrated in FIG. 10 and comprises a flushing assembly 210, including two primary flow lines 212 and 214, each connected in fluid communicating relation to a different one of two flush ports, 220 and 222. In addition, each of the primary flow lines 212 and 214, as well as their corresponding flush ports 220 and 222, are removably connected to the corresponding lumens 116 and 116′ of the catheter 16, independently of one another. More specifically, each of two connectors 150 is disposed and structured to independently connect and disconnect a different one of the primary flow lines 212 and 214 to a corresponding lumen 116 and 116′ of the catheter 16.

Still referring to FIG. 10, the assembly 210 also includes a primary flow restrictor assembly 228 comprising two safety flow restrictors 230 and 232, which may be in the form of exteriorly applied clamps, similar to the safety clamps or like flow restrictors 30″ and 32″ of the embodiment of FIG. 9. The assembly 210 also includes a secondary flow restrictor assembly 240 comprising two flow restrictors 242 and 244, which also may be in the form of exterior clamps, as set forth above. Therefore, and similar to the embodiments of FIGS. 1-7, a flushing orientation of the assembly 210 comprises the flow restrictors 230 and 232 of the primary flow restrictor assembly 228 being disposed in a closed position, thereby preventing the flushing fluid from pass into the catheter lumens 116 and 116′ and bloodstream of the patient. The flushing orientation of the assembly 210 is further defined by the flow restrictors 242 and 244 of the secondary flow restrictor assembly 240 being disposed in an open position, such as by being removed or otherwise oriented in a non-clamping relation to the flow ports 220 and 222. The opening of the flow restrictors 242 and 244 will result in the flush ports 220 and 222 being disposed in fluid communication with corresponding ones of the primary flow lines 212 and 214, concurrently to the primary flow lines 212 and 214 being isolated from the catheter lumens 116 and 116′.

As a result, the path of the flushing fluid within the assembly 210 of FIG. 10, when it is in the flushing orientation, comprises an entry of the flushing fluid through the open ends or injector caps 213 and 215 of the primary flow lines 212 and 214, as schematically represented at 46 and 48, and along the length of the primary flow lines 212 and 214. The path of flow of the flushing fluid will continue through the corresponding flush ports 212 and 214 and outwardly there-from through the exit ends 220′ and 222′, and the corresponding one-way valves 224 and 226 associated with the exit ends 220′ and 222′ of the flush ports 220 and 222.

Since many modifications, variations and changes in detail can be made to the described preferred embodiment of the invention, it is intended that all matters in the foregoing description and shown in the accompanying drawings be interpreted as illustrative and not in a limiting sense. For instance, it should be appreciated that the flushing assembly may be fixedly associated with or incorporated with a catheter, such as might occur during the manufacturing process associated with the catheter, or alternatively, produced as a stand alone device that may be interconnected with and removably attached to a catheter for use. Thus, the scope of the invention should be determined by the appended claims and their legal equivalents.

Now that the invention has been described,

What is claimed is:

1. An assembly for flushing primary flow lines associated with an intravascular catheter, said assembly comprising:
a first flush port and a second flush port each connected in fluid communicating relation with a different one of said primary flow lines,
a primary flow restrictor assembly disposed at a main junction of the intravascular catheter and said primary flow lines,
said primary flow restrictor assembly structured to regulate fluid flow between said primary flow lines and the intravascular catheter,
said primary flow restrictor assembly disposed in a closed position to define a flushing orientation of said first and second flush ports, and
said flushing orientation further comprising each of said first and second flush ports connected in fluid communication with corresponding ones of said primary flow lines and a concurrent flow path of flushing fluid from said primary flow lines out through said first and second flush ports.

2. An assembly as recited in claim 1 further comprising a secondary flow restrictor assembly disposed and structured to isolate fluid communication between each of said first and second flush ports and corresponding ones of said primary flow lines.

3. An assembly as recited in claim 2 wherein said secondary flow restrictor assembly comprises at least two flow restrictors each independently operable and disposed in flow restricting relation between a different one of said first and second flush ports and corresponding ones of said primary flow lines.

4. An assembly as recited in claim 1 wherein each of said first and second flush ports include an exit end and a one-way valve connected to said exit end, each of said one-way valves disposed and structured to restrict fluid flow into a corresponding one of said first and second flush ports through said exit ends.

5. An assembly as recited in claim 4 wherein each of said one-way valves is further disposed and structured to facilitate fluid flow out of said first and second flush ports through corresponding ones of said exit ends.

6. An assembly as recited in claim 5 wherein said secondary flow restrictor assembly comprises two independently operable flow restrictors each disposed in flow restricting relation between a different one of said first and second flush ports and corresponding ones of said primary flow lines.

7. An assembly as recited in claim 6 wherein said primary flow restrictor assembly comprises at least two safety valves each fixedly connected adjacent the main junction of the catheter, each of said two safety valves structured for selective orientation between an opened position and a closed position, each of said two safety valves disposed in flow regulating relation between a different one of said primary flow lines and a remainder of said catheter.

8. An assembly as recited in claim 7 wherein each of said two safety valves is structured and disposed in flow regulating relation between a different one of said flush ports and the remainder of said catheter.

9. An assembly as recited in claim 1 wherein said primary flow restrictor assembly comprises at least two safety flow restrictors each fixedly connected adjacent the main junction of the catheter, each of said two safety flow restrictors structured for selective disposition between an opened position and a closed position, each of said two safety flow restrictors disposed in flow regulating relation between a different one of said primary flow lines and a remainder of said catheter.

10. An assembly as recited in claim 9 wherein each of said two safety flow restrictors is structured and disposed in flow regulating relation between a different one of said flush ports and the remainder of said catheter.

11. An assembly as recited in claim 1 wherein each of said first and second flush ports and corresponding ones of said primary flow lines comprise a common, substantially Y-shaped junction.

12. An assembly as recited in claim 11 wherein said primary flow restrictor assembly comprises two safety flow restrictors, each of said safety flow restrictors disposed adjacent and in flow regulating relation to a different one of said Y-shaped junctions.

13. An assembly as recited in claim 11 wherein each of said flush ports comprises an elongated configuration extending outwardly from a corresponding one of said Y-shaped junctions into a connected engagement with a corresponding primary flow line.

14. An assembly as recited in claim 13 wherein each of said first and second flush ports are exteriorly connected to a corresponding one of said primary flow lines along at least a majority of the length of said first and second flush ports.

15. An assembly as recited in claim 13 wherein each of said first and second flush ports is exteriorly connected to a corresponding one of said primary flow lines intermediate a length of said first and second flush ports; a remainder of the length of each of said first and second flush ports being disposed in spaced relation to a corresponding one of said primary flow lines.

16. An assembly as recited in claim 11 wherein said first and second flush ports collectively define a dual lumen construction integrated into a common conduit.

17. An assembly as recited in claim 16 further comprising a one-way valve connected to an exit end of said dual lumen construction, said one-way valve disposed and structured to restrict fluid flow into a corresponding one of said first and second flush ports through said exit end.

18. An assembly as recited in claim 16 wherein each of said first and second flush ports include an exit end and a one-way valve connected thereto, each of said one-way valves disposed and structured to restrict fluid flow into a corresponding one of said first and second flush ports through corresponding ones of said exit ends.

19. An assembly as recited in claim 11 wherein said first and second flush ports comprise a common lumen; a one-way valve connected to an exit end of said common lumen, said one-way valve disposed and structured to restrict fluid flow into said common lumen through said exit end.

20. An assembly as recited in claim 1 wherein each of said primary flow lines and corresponding ones of said first and second flush ports are removably connected to a remainder of the catheter, independently of the other of said primary flow lines.

21. An assembly for concurrently flushing primary flow lines associated with a catheter, said assembly comprising:
a first flush port connected in fluid communication with one of said primary flow lines,
a second flush port connected in fluid communication with the other of said two primary flow lines,
a primary flow restrictor assembly comprising two safety flow restrictors each structured for selective orientation between a closed position and an opened position,
each of said two safety flow restrictors disposed in flow regulating relation between a different one of said flush ports and the remainder of the catheter,
each of said safety flow restrictors disposed in flow regulating relation between a different one of said primary flow lines and said catheter,
said two safety flow restrictors concurrently disposed in a closed position to define a flushing orientation of said first and second flush ports, and
said flushing orientation further comprising each of said first and second flush ports connected in fluid communication with corresponding ones of said primary flow lines and a concurrent path of flushing fluid flow from each of said primary flow lines toward and out through said first and second flush ports.

22. An assembly as recited in claim 21 further comprising a secondary flow restrictor assembly disposed and structured to isolate fluid communication between each of said first and second flush ports and corresponding ones of said primary flow lines.

23. An assembly as recited in claim 21 wherein said secondary flow restrictor assembly comprises two independently operable flow restrictors each disposed in flow restricting relation between a different one of said first and second flush ports and corresponding ones of said primary flow lines.

24. An assembly as recited in claim 21 wherein each of said first and second flush ports include an exit end and a one-way valve connected thereto, each of said one-way valves disposed and structured to restrict fluid flow into a corresponding one of said first and second flush ports through a corresponding one of said exit ends.

25. An assembly as recited in claim 24 wherein each of said one-way valves is further disposed and structured to facilitate fluid flow out of said first and second flush ports through respective ones of said exit ends.

26. An assembly for flushing primary flow lines associated with an intravascular catheter, said assembly comprising:
a bridging conduit disposed in interconnecting relation with said primary flow lines,
said bridging conduit disposed and structured to establish fluid communication with and between the primary flow lines,
a primary flow restrictor assembly structured to regulate fluid flow between said primary flow lines and the intravascular catheter,
a secondary flow restrictor assembly disposed on said bridging conduit and structured to regulate fluid flow between said primary flow lines,
said first and second primary flow restrictor assemblies respectively disposed in a closed position and an opened position to define a flushing orientation, and
said flushing orientation further comprising said primary flow lines disposed in fluid communication with one another via said bridging conduit and a concurrent flow path of flushing fluid into and through one of said primary flow lines, through said bridging conduit and out through the other of said primary flow lines.

27. An assembly as recited in claim 26 wherein said bridging conduit is fixedly connected to both of said primary flow lines.

28. An assembly as recited in claim 27 wherein said bridging conduit includes two opposite ends each fixedly connected to a different one of said primary flow lines.

29. An assembly as recited in claim 28 wherein said primary flow lines and said bridging conduit are removably connected to a remainder of the catheter.

30. An assembly as recited in claim 28 wherein said primary flow lines and said bridging conduit collectively define a substantially H-shaped configuration.

31. An assembly as recited in claim 26 wherein said primary flow lines are removably connected to a remainder of said catheter.
